(19) United States
(12) Patent Application Publication
(10) Pub. No.: US 2012/0253415 A1
(43) Pub. Date: Oct. 4, 2012

(54) SOUND CONTROL BODY SHAPING APPARATUS

(76) Inventor: Chun-Yuan HSU, Houlong Township (TW)

(21) Appl. No.: 13/167,994

(22) Filed: Jun. 24, 2011

(30) Foreign Application Priority Data
Mar. 28, 2011 (TW) 100205460

(51) Int. Cl. A61N 1/36

(52) U.S. Cl. 607/2

(57) ABSTRACT

A sound control body shaping apparatus includes a sound signal control conversion device, an audio device and an electrode transmission device. The sound signal input of the audio device is selected through a signal switch unit of the sound signal control conversion device. The frequency and signal volume of the input signal are converted into electrical energy for operation through a micro-computer frequency and signal detection unit. The operation result is transmitted to a voltage control circuit and a frequency control circuit of a signal control circuit unit so as to control the electrical energy and the frequency outputted to the electrode transmission device. The output electrical quantity and frequency of the electrical energy can be changed along with music and surrounding sound. The present invention has a better use function and provides various uses.

### BACKGROUND OF THE INVENTION

1. Field of the Invention

The present invention relates to a sound control body shaping apparatus, and more particularly to one applied to an electrode transmission device.

2. Description of the Prior Art

A conventional electrode transmission device comprises a controller and a plurality of adhesive electrodes. The adhesive electrodes are connected with the controller through conductive wires. The controller generates low-frequency current signal which is transmitted to the adhesive electrodes and then to the user's skin. There are some problems existing in the conventional electrode transmission device.

1. Insufficient use function: The controller controls the current intensity and the frequency to be transmitted to the adhesive electrodes. The current intensity and the frequency cannot be changed as desired, so the use function is insufficient.

2. Insufficient variation: The controller controls the current intensity and the frequency to be transmitted to the adhesive electrodes. The current intensity and the frequency cannot be adjusted to cooperate with music and external sound, so the variation of use is insufficient.

Accordingly, the inventor of the present invention has devoted himself based on his many years of practical experiences to solve these problems.

### SUMMARY OF THE INVENTION

The primary object of the present invention is to provide a sound control body shaping apparatus which comprises a sound signal control conversion device, an audio device and an electrode transmission device. The sound signal control conversion device comprises a signal switch unit, a micro-computer frequency and signal detection unit and a signal control circuit unit which are electrically connected to each other. The audio device is electrically connected with the sound signal control conversion device. The electrode transmission device is electrically connected with the sound signal control conversion device.

Thereby, the sound signal control conversion device is electrically connected with the audio device and the electrode transmission device. The sound signal input of the audio device is selected through the signal switch unit of the sound signal control conversion device. The frequency and signal volume of the input signal are converted into electrical energy for operation through the micro-computer frequency and signal detection unit. The display unit of the micro-computer frequency and signal detection unit is used to look up and control the data. The operation result is transmitted to the voltage control circuit and the frequency control circuit of the signal control circuit unit so as to control the electrical energy and the frequency outputted to the electrode transmission device. Furthermore, the signal switch unit of the sound signal control conversion device is provided with a wireless receiver at a front end thereof. The audio device is a wireless play device which has a wireless transmitter. The wireless transmitter connects with a wireless microphone or a wireless sounder. Through the wireless play device, the signal is transmitted to the wireless receiver of the sound signal control conversion device for subsequent operation and output. The output electrical quantity and frequency of the electrical energy can be changed along with music and surrounding sound. The present invention has a better use function and provides various uses.

### BRIEF DESCRIPTION OF THE DRAWINGS

FIG. 1 is a diagram according to a preferred embodiment of the present invention;

FIG. 2 is a diagram showing the sound signal control conversion device according to the preferred embodiment of the present invention;

FIG. 3 is a diagram showing the signal control circuit unit according to the preferred embodiment of the present invention;

FIG. 4 is a diagram showing the audio device according to the preferred embodiment of the present invention;

FIG. 5 is a diagram showing the wireless audio device according to the preferred embodiment of the present invention; and

FIG. 6 is a schematic view of the preferred embodiment of the present invention when in use.

### DETAILED DESCRIPTION OF THE PREFERRED EMBODIMENTS

Embodiments of the present invention will now be described, by way of example only, with reference to the accompanying drawings.

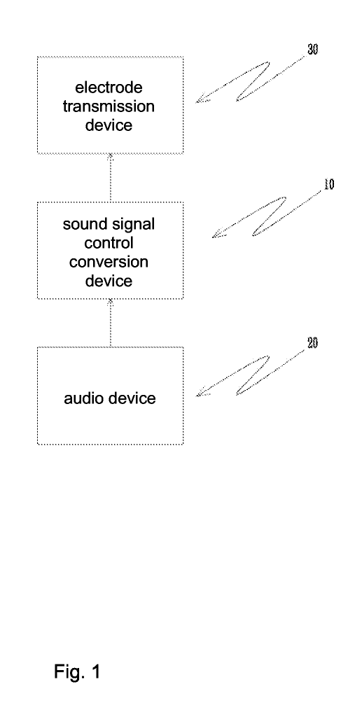

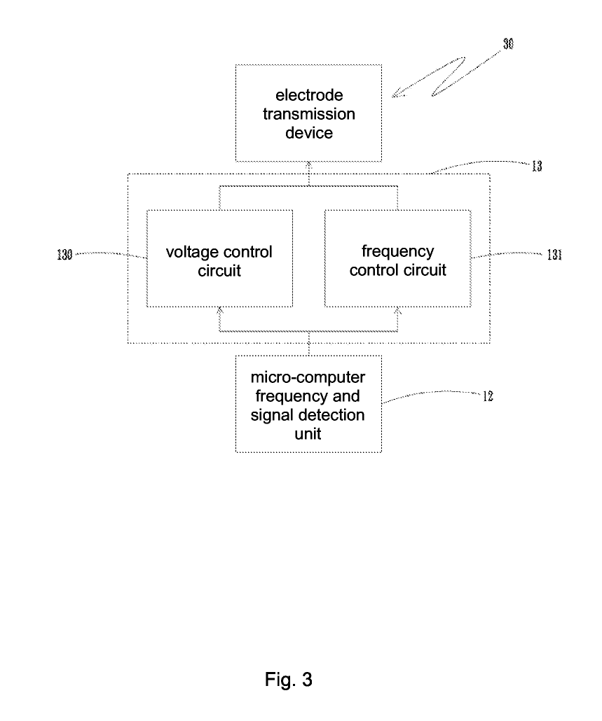

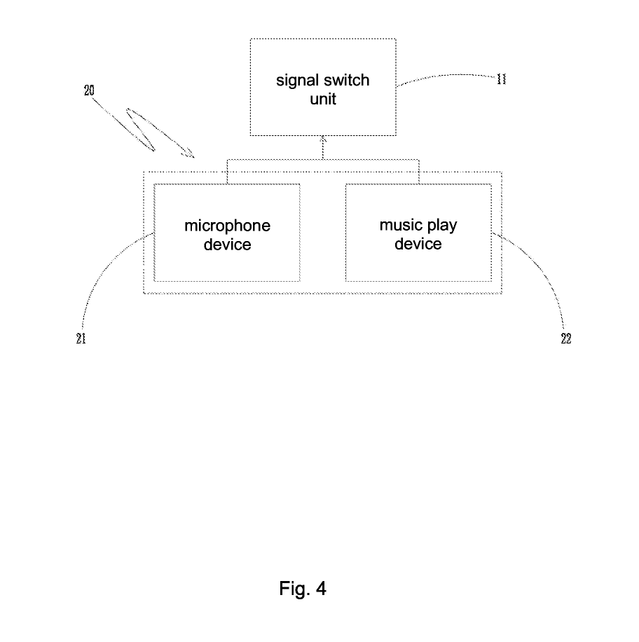

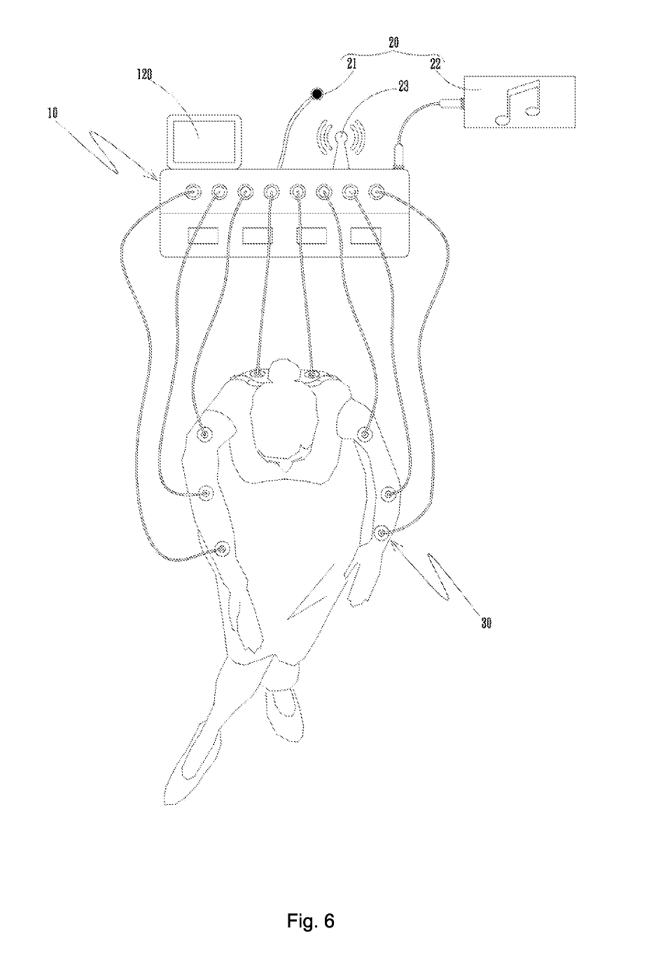

As shown in FIG. 1 through FIG. 4, and FIG. 6, the sound control body shaping apparatus of the present invention comprises a sound signal control conversion device (10), an audio device (20), and an electrode transmission device (30).

The sound signal control conversion device (10) comprises a signal switch unit (11), a micro-computer frequency and signal detection unit (12) and a signal control circuit unit (13) which are electrically connected to each other. The micro-computer frequency and signal detection unit (12) is connected with a display unit (120). The signal control circuit unit (13) comprises a voltage control circuit (130) and a frequency control circuit (131).

The audio device (20) is a microphone device (21) or a music play device (22). The audio device (20) is electrically connected with the sound signal control conversion device (10).

The electrode transmission device (30) is electrically connected with the sound signal control conversion device (10). The electrode transmission device (30) is a conductive electrode plate.

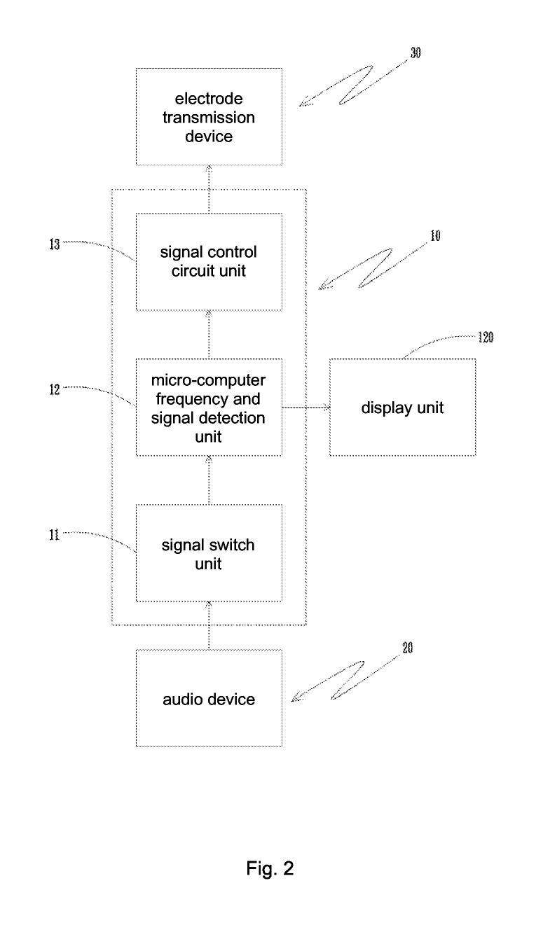

Referring to FIG. 2 through FIG. 4, the sound signal control conversion device (10) is electrically connected with the audio device (20) and the electrode transmission device (30). The sound signal input source of the audio device (20) is selected through the signal switch unit (11) of the sound signal control conversion device (10). The frequency and signal volume of the input signal are converted into electrical energy for operation through the micro-computer frequency and signal detection unit (12). The display unit (120) of the micro-computer frequency and signal detection unit (12) is used to look up and control the data. The operation result is transmitted to the voltage control circuit (130) and the frequency control circuit (131) of the signal control circuit unit (13) so as to control the electrical energy and the frequency outputted to the electrode transmission device (30) for subsequent operation and output. The output electrical quantity and frequency of the electrical energy can be changed along with music and surrounding sound. The present invention has a better use function and provides various uses.

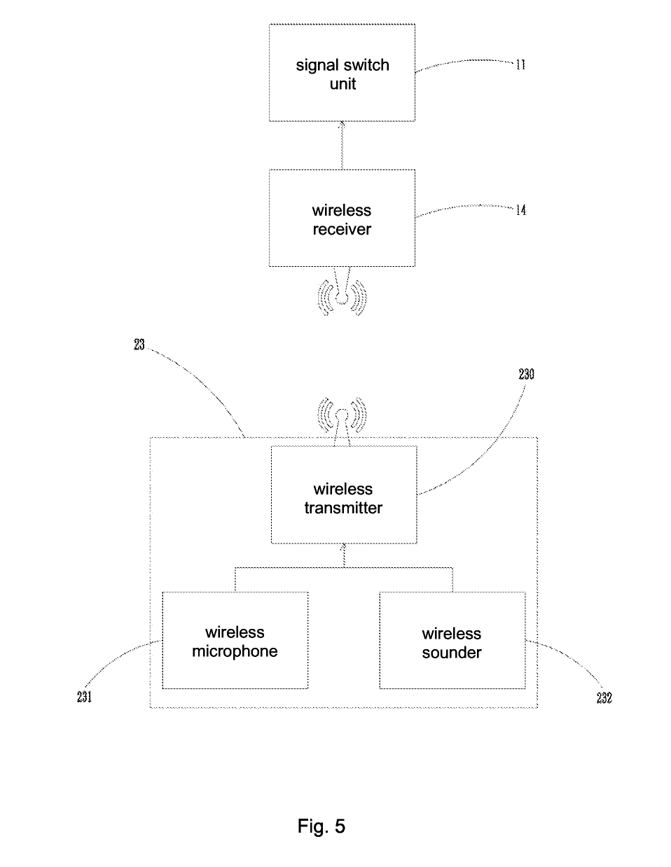

Referring to FIG. 2 and FIG. 5, the signal switch unit (11) of the sound signal control conversion device (10) is provided with a wireless receiver (14) at a front end thereof. The audio device (20) is a wireless play device (23) which has a wireless transmitter (230). The wireless transmitter (230) connects with a wireless microphone (231) or a wireless sounder (232). Through the wireless play device (23), the signal is transmitted to the wireless receiver (14) of the sound signal control conversion device (10) for subsequent operation and output. The output electrical quantity and frequency of the electrical energy can be changed along with music and surrounding sound. The present invention has a better use function and provides various uses.

The present invention has the following advantages.

1. Better use function: The sound signal control conversion device can connect with the microphone device, the music play device or the wireless play device of the audio device, and the output electrical quantity and frequency of the electrical energy can be changed along with music and surrounding sound. The present invention has a better use function.

2. Various uses: The sound signal input of the audio device is selected through the signal switch unit of the sound signal control conversion device. The frequency and signal volume of the input signal are converted into electrical energy for operation through the micro-computer frequency and signal detection unit. The display unit of the micro-computer frequency and signal detection unit is used to look up and control the data. The operation result is transmitted to the voltage control circuit and the frequency control circuit of the signal control circuit unit so as to control the electrical energy and the frequency outputted to the electrode transmission device for subsequent operation and output. The output electrical quantity and frequency of the electrical energy can be changed along with music and surrounding sound. The present invention provides various uses.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A sound control body shaping apparatus, comprising:
a sound signal control conversion device, the sound signal control conversion device comprising a signal switch unit, a micro-computer frequency and signal detection unit and a signal control circuit unit which are electrically connected to each other;
an audio device, the audio device being electrically connected with the sound signal control conversion device; and
an electrode transmission device, the electrode transmission device being electrically connected with the sound signal control conversion device.

2. The sound control body shaping apparatus as claimed in claim 1, wherein the micro-computer frequency and signal detection unit is connected with a display unit.

3. The sound control body shaping apparatus as claimed in claim 1, wherein the signal control circuit unit comprises a voltage control circuit and a frequency control circuit.

4. The sound control body shaping apparatus as claimed in claim 1, wherein the audio device is a microphone device.

5. The sound control body shaping apparatus as claimed in claim 1, wherein the audio device is a music play device.

6. The sound control body shaping apparatus as claimed in claim 1, wherein the signal switch unit of the sound signal control conversion device is provided with a wireless receiver at a front end thereof, and the audio device is a wireless play device.

7. The sound control body shaping apparatus as claimed in claim 6, wherein the wireless play device has a wireless transmitter, the wireless transmitter connecting with a wireless microphone, a signal being transmitted to the wireless receiver of the sound signal control conversion device through the wireless play device.

8. The sound control body shaping apparatus as claimed in claim 7, wherein the wireless transmitter connects with a wireless sounder, a signal being transmitted to the wireless receiver of the sound signal control conversion device through the wireless play device.
